(19) United States
(12) Patent Application Publication
(10) Pub. No.: US 2003/0149013 A1
(43) Pub. Date: Aug. 7, 2003

(54) Methods of treating bacterial infections in dogs and cats

(76) Inventors: Simon E. Blanchflower, Sandwich (GB); Brian S. Bronk, Gales Ferry, CT (US)

Correspondence Address: PFIZER INC., PATENT DEPARTMENT, MS8260-1611, EASTERN POINT ROAD, GROTON, CT, 06340, US

(21) Appl. No.: 10/292,145

(22) Filed: Nov. 12, 2002

Related U.S. Application Data
(60) Provisional application No. 60/359,961, filed on Feb. 27, 2002.
(60) Provisional application No. 60/398,933, filed on Jul. 26, 2002.

(30) Foreign Application Priority Data
Dec. 21, 2001 (GB) 0130694.2

(51) Int. Cl. A61K 31/545

(52) U.S. Cl. 514/202

(57) ABSTRACT

The invention described herein relates to the treatment of a range of bacterial infections in companion animals, in particular cats and dogs, with a β-lactam derivative, compound of Formula I. The invention is also directed to pharmaceutical compositions of a compound of Formula I.

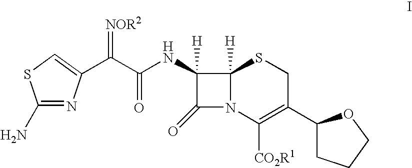

### FIELD OF INVENTION

The invention described herein relates to the treatment of a range of bacterial infections in companion animals, in particular cats and dogs, with a β-lactam derivative, compound of Formula I. The invention is also directed to pharmaceutical compositions of a compound of Formula I.

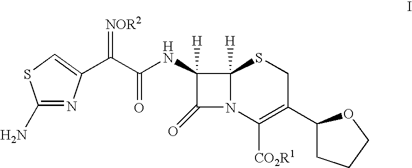

### BACKGROUND OF INVENTION

β-Lactam antibiotics, such as penicillins and cephalosporins, have been known for some time and are the subject of many review articles. See, for example, Harvey, R. G., Hunter, P. A. The properties and use of penicillins in the veterinary field, with special reference to skin infections in dogs and cats. Veterinary Dermatology, 10:3, September 1999; Mason, I. S., Kietzmann, M. Cephalosporins—pharmacological basis of clinical use in veterinary dermatology. Veterinary Dermatology, 10:3, September 1999; and the references therein.

A number of cephalosporin derivatives, including several incorporating a cyclic ether moiety at the 3-position, were disclosed in International Patent Application publication number WO 92/01696 and by Bateson et al in The Journal of Antibiotics, February 1994, vol.47, no.2, at pages 253-256. Various mouse data are also disclosed in the latter paper. A process for preparing the cephalosporins is described in EP1178049A1. These publications are herein incorporated in their entirety.

### SUMMARY OF INVENTION

In one aspect, the invention is directed to a pharmaceutical composition for treating periodontal diseases in a dog or cat caused by bacterial infections comprising a therapeutically effective amount of a compound of Formula I,

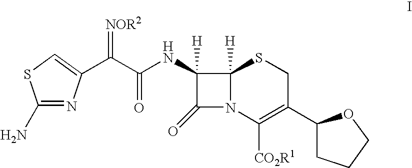

wherein R¹ is H, a pharmaceutically acceptable cation salt, or an in vivo hydrolysable group capable of generating CO₂H, and R² is C₁₋₄ alkyl, and a pharmaceutical diluent or carrier.

In a preferred embodiment, the pharmaceutically acceptable cation salt is Na⁺, K⁺ or Li⁺.

In a second aspect, the invention is directed to a pharmaceutical composition for treating opportunistic bacterial infections resulting from (a) a compromised or diminished immunological system, (b) Feline Immunodeficiency Virus (FIV) or (c) cancer chemotherapy in or on a dog or cat comprising a therapeutically effective amount of a compound of Formula I,

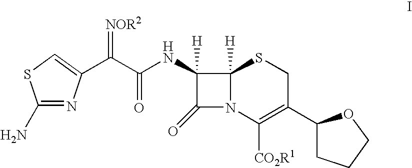

wherein R¹ is H, a pharmaceutically acceptable cation salt, or an in vivo hydrolysable group capable of generating CO₂H, and R² is C₁₋₄ alkyl, and a pharmaceutical diluent or carrier.

In a third aspect, the invention is directed to a pharmaceutical composition for treating a disease or condition caused by a bacterial infection in a dog or cat comprising a therapeutically effective amount of a compound of Formula I,

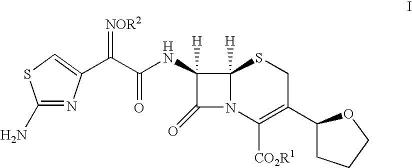

wherein R¹ is H, a pharmaceutically acceptable cation salt, or an in vivo hydrolysable group capable of generating CO₂H, and R² is C₁₋₄ alkyl, and a pharmaceutical diluent or carrier, with the proviso that if the pharmaceutically acceptable cation salt is Na, Li or K, R² is not methyl.

In a preferred embodiment, the disease or condition is a skin, soft tissue or urinary tract bacterial infection.

In another embodiment, R¹ is H, an alkaline metal, an alkaline earth metal, ammonium, benzyl, p-methoxybenzyl, benzoylmethyl, p-nitrobenzyl, 4-pyridylmethyl, 2,2,2-trichloroethyl, 2,2,2-tribromoethyl, t-butyl, t-amyl, allyl, diphenylmethyl, triphenylmethyl, adamantyl, 2-benzoyloxyphenyl, 4-methylthiophenyl, tetrahydrofur-2-yl, tertahydropyran-2-yl, pentachlorophenyl, acetonyl, p-toluenesulphonylethyl, methoxymethyl, a silyl, stannyl or phosphorus-containing group, an oxime radical of formula —N═CHR³ where R³ is aryl or heterocyclyl, or an in vivo hydrolysable ester radical.

In a preferred embodiment, the compound of formula I is the Z-isomer.

In another preferred embodiment, R¹ is H, Na⁺ or CH₂OCOC(CH₃)₃).

In a more preferred embodiment, R¹ is H or CO₂CH₂OCOC(CH₃)₃) and R² is methyl.

In another embodiment, the pharmaceutical composition further comprises one or more agents used in the treatment or prophylaxis of disease or in the reduction or suppression of symptoms of such disease.

In a preferred embodiment, one or more of the agents are selected from antiparasitics, antihistamines, antifungals, antibacterials, anti-inflammatories, steroids, antipruritic agents,dietary supplements or emollients.

In a further preferred embodiment, the antiparasitics are selected from arylpyrazoles, avermectins, milbemycins, organophosphates or pyrethroids; the antihistamines are selected from chlorpheniramine, trimeprazine, diphenhydramine or doxylamine; the antifungals are selected from fluconazole, ketoconazole, itraconazole, griseofulvin or amphotericin B; the antibacterials are selected from enroflaxacin, marbofloxacin, ampicillin or amoxycillin; the anti-inflammatories are selected from prednisolone, betamethasone, dexamethasone, carprofen or ketoprofen; and the dietary supplement is gamma-linoleic acid.

In a fourth aspect, the invention is directed to a method of treating periodontal disease or an opportunistic bacterial infection in or on a dog or cat, resulting from (a) a compromised or diminished immunological system, (b) Feline Immunodeficiency Virus (FIV) or (c) cancer chemotherapy in or on a comprising administrating a therapeutically effective amount of a compound of Formula I

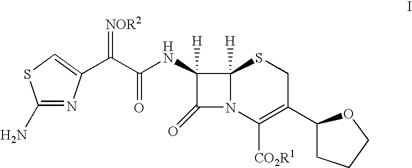

wherein R¹ is H, a pharmaceutically acceptable cation salt, or an in vivo hydrolysable group capable of generating CO₂H, and R² is C₁₋₄ alkyl, effective in treating such a condition.

In a preferred embodiment, the pharmaceutically acceptable cation salt is Na⁺, K⁺ or Li⁺.

In a fifth aspect, the invention is directed to a method of treating a disease or condition caused by a bacterial infection in or on a dog or cat comprising administrating a therapeutically effective amount of a compound of Formula I,

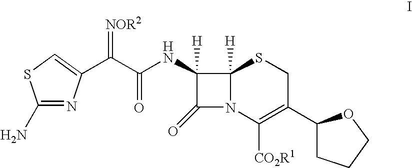

wherein R¹ is H, a pharmaceutically acceptable cation salt, or an in vivo hydrolysable group capable of generating CO₂H, and R² is C₁₋₄ alkyl, and a pharmaceutical diluent or carrier, with the proviso that if the pharmaceutically acceptable cation salt is Na, Li or K, R² is not methyl, effective in treating such a condition.

In a preferred embodiment, the disease or condition is a skin, soft tissue or urinary tract bacterial infection.

In a more preferred embodiment, the composition is administered to the dog or cat in a single dose.

In another preferred embodiment, the administration is subcutaneous.

In a preferred embodiment, the therapeutically effective amount of the compound of Formula I is 4 mg/kg to 12 mg/kg.

In a more preferred embodiment, the method provides a duration of treatment activity of at least five days against susceptible pathogens.

In a preferable embodiment, the duration is at least 7 days.

In another embodiment, the method of treating a disease or condition caused by a bacterial infection in or on a dog or cat comprises administrating a therapeutically effective amount of the pharmaceutical composition described above.

In a sixth aspect, the invention is directed to a kit for treatment or prevention of a bacterial infection or a condition caused or complicated by a bacterial infection, in or on a dog or cat, comprising:

a) a pharmaceutical composition as described above; and

b) instructions describing a method of using the pharmaceutical composition to treat a bacterial infection or a condition caused or complicated by a bacterial infection, in or on a dog or cat.

In a seventh aspect, the invention is directed to a method for increasing acute or chronic injection-site toleration in a dog or a cat, comprising administering to a dog or a cat in need thereof a single dose of a therapeutically effective amount of a Formula I

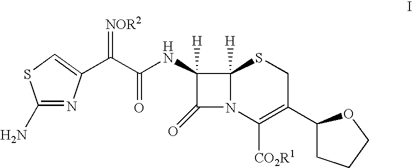

wherein R¹ is H, a pharmaceutically acceptable cation salt, or an in vivo hydrolysable group capable of generating CO₂H, and R² is C₁₋₄ alkyl, effective in treating such a condition.

The term “ammonium” as used herein means an ammonium moiety optionally substituted with C₁₋₆ alkyl, optionally substituted by OH; or C₅₋₇ cycloalkyl groups. For example, lower alkylamines may be triethylamine, hydroxy-lower alkylamines such as 2-hydroxyethylamine, bis-(2-hydroxyethyl) amine or tris(2-hydroxyethyl)-amine, cycloalkylamines such as dicyclohexylamine or with procaine, dibenzylamine, N,N-dibenzylethylene-diamine, 1-ephenamine, N-methylmorpholine, N-ethylpiperidine, N-benyzl-β-phenethylamine, dehydroabietylamine, N,N′-bisdehydro-abietylamine, ethylenediamine, or bases of the pyridine type such as pyridine, collidine, or quionoline, or other amines which have been used to form salts with known penicillins and cephalosporins. Other useful salts include lithium and silver salts. Salts within compounds of Formula I may be prepared by salt exchange in a conventional manner.

The term “in vivo hydrolysable ester group” means a pharmaceutically acceptable ester group that readily breaks down in the human body to leave the parent acid or its salt. Suitable ester groups of the type include those described in Bateson et al. (EP0540609B1), hereby incorporated by reference in its entirety. In particular, examples of suitable in vivo hydrolysable ester groups include acyloxyalkyl groups such as acetoxymethyl, pivaloyloxymethyl, α-acetoxyethyl, α-pivaloyloxyethyl, 1-(cyclohexylcarbonyloxy)prop-1-yl, and (1-aminoethyl)carbonyloxymethyl; alkoxycarbonyloxyalkyl groups, such as ethoxycarbonyloxymethyl, α-ethoxycarbonyloxyethyl and propoxycarbonyloxyethyl; dialkylaminoalkyl, especially di-loweralkylamino alkyl groups such as dimethylaminomethyl, dimethylaminoethyl, diethylaminomethyl or diethylaminoethyl; 2-(alkoxycarbonyl)-2-alkenyl groups such as 2-(isobutoxycarbonyl)pent-2-enyl and 2-(ethoxycarbonyl)but-2-enyl; lactone groups such as phthalidyl and dimethoxyphthalidyl; and esters linked to a second β-lactam antibiotic or to a β-lactamase inhibitor. Preferably, the in vivo hydrolysable ester group is the pivaloyloxymethyl ester.

The term “pharmaceutically acceptable salts” of the carboxy group of the compound of Formula I include metal salts, e.g. aluminum, alkali metal salts such as sodium or potassium, especially sodium, alkaline earth metal salts such as calcium or magnesium, and ammonium or substituted ammonium salts.

The term “therapeutically effective amount” as used herein means the dose of a compound of Formula I effective in treating bacterial infections. The dose may vary depending upon the dog or cat patient, but generally is about 0.01 to 100 mg/kg of the subject animal's weight.

### DESCRIPTION OF INVENTION

It was determined that the compounds of Formula I, exhibit an unexpectedly long half-life in dogs and cats, especially in view of comparable antibiotics. For example, Table I lists well-known antibiotics and their respective half-lives in different mammals, such as in mice, rats, dogs and cats.

TABLE I
Half-life of Known Antibiotics

| Compound | Mouse t_{1/2} (h) | Rat t_{1/2} h | Canine t_{1/2} h |
|---|---|---|---|
| Cefpodoxime | 0.68 (PO) | 1.4 (PO) | 2.4 (PO) |
| Ampicillin | | 0.84 (IM) | 0.64 (IM) |
| Cefamandole | | 0.5 (IM) | 0.82 (IV) |
| Cefazolin | | 0.66 (IM) | 1.11 (IV) |
| Cefuroxime | 0.32 (SC) | 0.4 (IM) | 0.93 (IM) |
| Cephalordine | | 0.5 (IM) | 0.97 (IM) |
| Cephalothin | 0.208 (IM) | 0.4 (IM) | 1.06 (IM) |
| Cephadrine | | 0.82 (PO) | 3.64 (P0) |
| Erythromycin | 0.65 (IV) | 1.27 (IV) | 1.72 (IV) |
| Oleandomycin | 0.7 (IV) | 0.93 (IV) | 1.53 (IV) |
| Tylosin | | 0.4 (IV) | 1.24 (IV) |
| (Cefpodoxime data from “Abstracts of the 1996 ICAAC”, Abstract 593. All other data compiled from: “CRC Handbook of Comparative Pharmacokinetics and Residues of Veterinary Antimicrobials”, J. Edmond Riviere; Arthur L. Craigmill, | | | |
| # Stephen F. Sundlof CRC Press 1991; Routes of administration: “IV”-intravenous; “IM” =intramuscular; “PO” = per os; “SC” = subcutaneous) | | | |

Following administration of the compound of Formula I, wherein R¹ is COC(CH₃)₃), R² is methyl and the oxime ether is in the Z-configuration, the half-life in the mouse and rat were determined, respectively, to be about 2.2 hours and about 3.9 hours, after per os administration. Unexpectedly, however, in dogs and cats, the half-life was in both cases dramatically increased, as is set forth in Table 2 below.

TABLE 2
Half-life of compound of formula I in Dogs and Cats

| | | | Formula I: Z- | Half- |
|---|---|---|---|---|
| Species | Route | Dose^{[1]} | (methyl oxime ether), R¹⁼ | life |
| Dog | IV | 1 mg/kg | Na (compound IA) | 6.9 |
| | | | | days |
| Dog | SC | 8 mg/kg | Na (compound IA) | 4.7 |
| | | | | days |
| Dog | PO | 1 mg/kg | pivaloyloxymethyl | 6.0 |
| | | | | days |
| | | | (compound IB) | |
| Cat | SC | 8 mg/kg | Na (compound IA) | 6.3 |
| | | | | days |

Note [1] Dose expressed as corresponding free acid: i.e. R¹ = H. Concentrations measured with respect to the free acid.

### EXPERIMENTAL DETAILS

1. Pharmacokinetics

### Experiment 1: Intravenous Dog

A male dog was dosed intravenously with an aqueous solution of Compound I. Blood plasma was sampled at times up to 28 days post dosing. Plasma samples were extracted and assayed to determine the concentration by both bioassay and High Pressure Liquid Chromatography (“HPLC”) as follows:

1 mL of plasma (or standards of spiked dog plasma) were acidified to a pH of less than 3 with hydrochloric acid, then shaken with 26 mL of ethyl acetate. The layers were separated by centrifugation. 22 mL of the organic layer was transferred into a fresh container and 2.0 mL of 0.1 M phosphate buffer, pH of 7.0, was added. After shaking and centrifugation, the aqueous phase was recovered and assayed. Following processing, samples (and standards) were assayed by hole-in-the-plate microbiological bioassay on large plates (200 mL Mueller Hinton agar) seeded with M. luteus. Samples were also assayed by HPLC (μBondapk —C18 column eluted with acetonitrile—0.05M sodium acetate pH 5.0, 15:85, with UV detection at 256 nm). Good agreement was obtained between the two assay methods, and the half-life was calculated from bioassay results using standard pharmacokinetic methods.

### Experiment 2: Subcutaneous Dog

Two dogs were dosed with compound of Formula I by subcutaneous injection. Blood plasma was sampled at times up to 28 days post-dosing. Plasma samples and appropriate standards were prepared by deproteination by the addition of an equal volume of acetonitrile and centrifugation (3000 r.p.m. for 10 minutes). Supernatant was assayed by a specific HPLC method to determine the concentration μBondapk —C18 column eluted with acetonitrile—0.05M sodium acetate pH 5.0, 15:85, at 1.0 mL/min with UV detection at 256 nm). Pharmacokinetic parameters were calculated using the program PCNONLIN.

### Experiment 3: per os Dog

Six dogs were dosed orally with the pivaloyloxymethyl-ester pro-drug, compound of Formula IB, and the resulting plasma concentrations were determined by both bioassay and HPLC. Following dosing, blood plasma was sampled up to 696 hours (29 days). Plasma samples and appropriate standards (1 mL) were first acidified to a pH less than 3.0 with hydrochloric acid then shaken with 30 mL ethyl acetate. The layers were separated by centrifugation then 25 mL of the organic layer was removed. 2 mL of 0.1M phosphate buffer pH 7.0 was added to the ethyl acetate and shaken to effect a back extraction. After separation of the layers, the aqueous phase was removed and used for the assays. Following processing, samples (and standards) were assayed by hole-in-the-plate microbiological bioassay on large plates (200 mL Mueller Hinton agar) seeded with M. luteus. Samples were also assayed by HPLC (μBondapk —C18 column eluted with acetonitrile—0.05M sodium acetate pH 5.0, 15:85, at 1.5 mL/min with UV detection at 256 nm). There was good agreement between the two assay methods (r=0.9716); the half-life was calculated from the bioassay data.

### Experiment 4: Subcutaneous Cat

Four cats were dosed at 8 mg/kg by subcutaneous injection of Compound I. Blood samples were taken at intervals to 35 days post-dosing and the plasma assayed to determine the concentration of the corresponding free acid by HPLCIMS/MS. Plasma samples (100 mL) were aliquoted into centrifuge tubes, then 400 mL of acetonitrile was added. Following vortexing (60 sec.) and centrifugation (20,800×g for 10 minutes), 0.450 mL of the supernatant was transferred into clean centrifuge tubes, and evaporated to dryness at approximately 50° C. under N₂. Dried samples were reconstituted in 0.100 mL of mobile phase (15/85 v/v acetonitrile/10 mM HCO₂NH₄, pH 3.0), vortexed for 1 minute, centrifuged at 3,000 rpm for 2 minutes, and transferred to an autosampler vial. Single replicates of plasma were analyzed by LC-MS/MS for concentration of compound. Sample analysis was performed on a SCIEX API 365 or 3000 HPLC/MS/MS system. The column effluent was connected to a Turbo-ionspray source set at 4500 V. The collision gas was set to a value of 3. Positive ions were generated in the source and sampled through an orifice into the quadrupole mass filter. The mass spectrometer was adjusted to monitor the precursor and product ions as follows: m/z 454.0->m/z 241.0. Half-life was calculated using pharmacokinetic program WINNONLIN v2.1 and determined to be 8.39+/−0.97 days.

2. Efficacy

In an experimentally induced skin infection model study, five out of six dogs had complete clearance of Staphylococcus intermedius 15 days after a single administration of 8 mg/kg of the Compound I.

In a separate study, following a single administration of 8 mg/kg Compound I to healthy dogs, there was a significant reduction of the populations of pathogenic Staphylococci for four weeks compared to non-treated control animals.

In an experimentally induced abscess model study in cats, there was a substantial reduction in the numbers of Pasteurella multocida, Clostridium perfringens, and Bacteriodes fragilis bacteria 14 days after a single administration of 8 mg/kg Compound I

The above half-life results, together with the potency of the compounds of Formula I, demonstrates that one administration of an equivalent of about 4-12 mg/kg of Compound I, (e.g. Na salt of compound of Formula I), given by injection (e.g. intramuscularly, subcutaneously or intravenously), to a cat or dog would advantageously provide an efficacious concentration for 7-21 days. This represents a novel and very convenient treatment regime for veterinary practitioners and cat and dog owners alike.

It will be noted that the compounds of Formula I have the 2S-stereochemistry in the furan moiety, also referred to as the Z isomer of the “oxime ether”. The invention is not, however, limited to uses of the pure 2S-enantiomer, and can be effected with efficacious mixtures of the 2S- and 2R-enantiomer, for example, as a racemic mixture (i.e. 1:1 2S:2R). The enantiomers may be obtained by classical resolution techniques well-known in the art, or by stereoselective synthesis. Enriched mixtures may be obtained by partial resolution, or partially stereoselective synthesis, or by admixing known amounts of known enantiomeric purity.

Preferably the proportion of 2S-enantiomer in any mixture of 2S and 2R enantiomers, used in accordance with this invention, is at least 20%.

More preferably the proportion is in the range 50%-100%.

Most preferably, the compound is present as a racemic mixture (i.e. 1:1 2S:2R), or as the substantially pure 2S isomer.

The compounds of Formula I may be administered by any convenient means for administering an antibiotic, such means being well-known in the art. The compounds could be administered orally (e.g. as a prodrug comprising a hydrolysable carboxy-protected ester moiety such as a pivaloyloxymethyl ester moiety) or by any parenteral route such as topically or by injection, in the form of pharmaceutical preparations comprising the compound, optionally in the form of a non-toxic organic, or inorganic, acid, or base, addition salt, or prodrug, in a pharmaceutically acceptable dosage form. Depending upon the disorder and patient to be treated, as well as the route of administration, the compositions may be administered at varying doses and times.

While it is possible to administer a compound of the invention directly without any formulation, the compounds are preferably employed in the form of a pharmaceutical (including veterinary) formulation comprising a pharmaceutically (including veterinary) acceptable carrier, diluent or excipient and a compound of Formula I. The carrier, diluent or excipient may be selected with due regard to the intended route of administration and standard pharmaceutical/veterinary practice. Pharmaceutical (including veterinary) compositions comprising the compounds of the invention may contain from about 0.1 percent by weight to about 90.0 percent by weight of the active ingredient.

The methods by which the compounds may be administered for veterinary use include oral administration (for example of a prodrug or salt of formula I by capsule, bolus, tablet, powder or drench, elixir, solution, paste, suspension, medicated feed or drinking water, or buccally, or sublingually, that may contain flavouring, palatable or colouring agents, and which may be prepared for immediate, delayed-, modified-, sustained-, pulsed-, or controlled-release; topical administration as an ointment, a pour-on, spot-on, dip, spray, mousse, shampoo, collar or powder formulation, ear tags; by injection (e.g., subcutaneously, intramuscularly or intravenously); as an implant. Such formulations may be prepared in a conventional manner in accordance with standard veterinary practice.

For example, formulations of the compounds of Formula I may contain physiologically-acceptable preservatives (such as those of the “paraben” family, buffering agents, solvents (such as water), and other ingredients used in accordance with standard veterinary practice and with the intended mode of administration.

The formulations will vary with regard to the weight of active compound contained therein, depending on the species, variety, etc. of animal to be treated, the severity and type of infection and the body weight of the animal. For parenteral, topical and oral administration, typical dose ranges of the active ingredient are 0.01 to 100 mg per kg of body weight of the animal. Preferably the range is 1 to 20 mg per kg, more preferably 4 to 12 mg per kg.

In any event, the veterinary practitioner, or the skilled person, will be able to determine the actual dosage which will be suitable for an individual patient, which may vary with the species, age, weight and response of the particular patient, as well as the bacterial species involved. The above dosages are exemplary of the average case; there can, of course, be individual instances where higher or lower dosage ranges are merited, and such are within the scope of this invention.

As an alternative for treating animals, the compounds may be administered with the animal feedstuff or drink, and for this purpose a concentrated feed or drink additive or premix may be prepared for mixing with the normal animal feed or drink.

Compounds of Formula I may be administered either alone or in combination with one or more agents used in the treatment or prophylaxis of disease or in the reduction or suppression of symptoms. Examples of such agents, include but are not limited to, antiparasitics, (e.g. arylpyrazoles such as fipronil, lufenuron, imidacloprid, avermectins (e.g. abamectin, ivermectin, doramectin and selamectin), milbemycins, organophosphates and pyrethroids); antihistamines (e.g. chlorpheniramine, trimeprazine, diphenhydramine and doxylamine); antifungals (e.g.fluconazole, ketoconazole, itraconazole, griseofulvin and amphotericin B); antibacterials (e.g. enroflaxacin, marbofloxacin, ampicillin and amoxycillin); anti-inflammatories (e.g. prednisolone, betamethasone, dexamethasone, carprofen and ketoprofen); steroids or other antipruritic agents; dietary supplements (e.g. gamma-linoleic acid); and emollients. Therefore, the invention further provides for uses, etc., of compounds of Formula I and one or more selected compounds from the above list as a combined preparation for simultaneous, separate or sequential use in the treatment of diseases or conditions according to the invention.

The skilled person will also appreciate that compounds of the invention may be taken as a single dose or on an “as required” basis (i.e. as needed or desired).

Preferred administration routes are intravenous, subcutaneous or intramuscular injection or topical or oral administration. For injection, preferably a solution of a compound of Formula I where R² is Na, e.g. the compound IA, in water is injected either intravenously, subcutaneously or intramuscularly. Especially preferred is subcutaneous injection. For oral administration, an oral formulation of a prodrug is preferred, such as a formulation of a compound of formula I where R² is pivaloyloxymethyl, such as the compound IB.

The compounds of Formula I are potent broad spectrum antibiotics, and as such can be used to treat infections and conditions caused by a wide range of bacteria. Particularly of interest are: skin and soft tissue infections, urinary tract and periodontal infections. More particularly of interest are conditions or infections caused by or complicated by Gram positive and/or Gram negative bacteria, such as canine pneumonia, feline pneumonia, canine pyoderma, feline pyoderma, pasteurellosis, pneumonia, otitis media, sinusitus, bronchitis, tonsillitis, and mastoiditis associated with infection by Staphylococcus spp. (Staphylococcus intermedius, Staphyloccus aureus), Escherichia coli, Streptococcus spp. (Beta Hemolytic Streptococcus spp.), Pasteurella multocida, Bacteriodes spp., Fusobacterium spp., Porphyromonas spp., Prevotella spp., Peptostreptococcus spp., and Clostridium spp., uncomplicated skin and soft tissue infections, abscesses, osteomyelitis, and puerperal fever associated with infection by Staphylococcus aureus, S. intermedius, coagulase-positive staphylococci, S. epidermidis, S. hemolyticus, Streptococcus ssp, Streptococcal groups C-F (minute-colony streptococci), viridans streptococci, uncomplicated acute urinary tract infections associated with infection by Staphylococcus ssp or E. coli.; odontogenic infection associated with infection by viridans streptococci; urinary tract infection in dogs and cats associated with infection by E. coli., skin and soft tissue infections in dogs and cats associated with infection by Staph. epidermidis, Staph. intermedius, coagulase neg. Staph. or P. multocida; infections of the oral cavity in dogs and cats associated with infection by Alcaligenes spp., Bacteroides spp., Clostridium spp., Enterobacter spp., Eubacterium, Peptostreptococcus, Porphyromonas, or Prevotella.

1. A pharmaceutical composition for treating periodontal diseases in a dog or cat caused by bacterial infections comprising a therapeutically effective amount of a compound of Formula I, 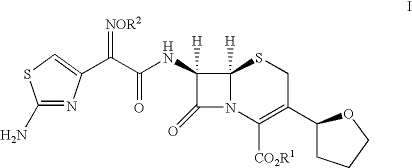
wherein R¹ is H, a pharmaceutically acceptable cation salt, or an in vivo hydrolysable group capable of generating CO₂H, and R² is C₁₋₄ alkyl, and a pharmaceutical diluent or carrier.

2. A pharmaceutical composition according to claim 1, wherein the pharmaceutically acceptable cation salt is Na⁺, K⁺ or Li⁺.

3. A pharmaceutical composition for treating opportunistic bacterial infections resulting from (a) a compromised or diminished immunological system, (b) Feline Immunodeficiency Virus (FIV) or (c) cancer chemotherapy in or on a dog or cat comprising a therapeutically effective amount of a compound of Formula I, 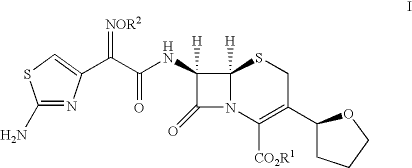
wherein R¹ is h, a pharmaceutically acceptable cation salt, or an in vivo hydrolysable group capable of generating CO₂H, and R² is C₁₋₄ alkyl, and a pharmaceutical diluent or carrier.

4. A pharmaceutical composition for treating a disease or condition caused by a bacterial infection in a dog or cat comprising a therapeutically effective amount of a compound of Formula I, 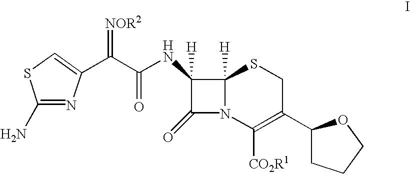
wherein R¹ is H, a pharmaceutically acceptable cation salt, or an in vivo hydrolysable group capable of generating CO₂H, and R² is C₁₋₄ alkyl, and a pharmaceutical diluent or carrier, with the proviso that if the pharmaceutically acceptable cation salt is Na, Li or K, R² is not methyl.

5. A pharmaceutical composition according to claim 4, wherein the disease or condition is a skin, soft tissue or urinary tract bacterial infection.

6. A pharmaceutical composition according to claims 1, 2, 3, 4 or 5, wherein R¹ is H, an alkaline metal, an alkaline earth metal, ammonium, benzyl, p-methoxybenzyl, benzoylmethyl, p-nitrobenzyl, 4-pyridylmethyl, 2,2,2-trichloroethyl, 2,2,2-tribromoethyl, t-butyl, t-amyl, allyl, diphenylmethyl, triphenylmethyl, adamantyl, 2-benzoyloxyphenyl, 4-methylthiophenyl, tetrahydrofur-2-yl, tertahydropyran-2-yl, pentachlorophenyl, acetonyl, p-toluenesulphonylethyl, methoxymethyl, a silyl, stannyl or phosphorus-containing group, an oxime radical of formula —N═CHR³ where R³ is aryl or heterocyclyl, or an in vivo hydrolysable ester radical.

7. A pharmaceutical composition according to claim 6, wherein the compound of formula I is the Z-isomer.

8. A pharmaceutical composition according to claim 7, wherein R¹ is H, Na⁺ or CH₂OCOC(CH₃)₃).

9. A pharmaceutical composition according to claims 1, 2 or 3, wherein R¹ is H or CO₂CH₂OCOC(CH₃)₃) and R² is methyl.

10. A pharmaceutical composition according to claims 1, 2, 3, 4 or 5, further comprising one or more agents used in the treatment or prophylaxis of disease or in the reduction or suppression of symptoms of such disease.

11. A pharmaceutical composition according to claim 10, wherein one or more of the agents are selected from antiparasitics, antihistamines, antifungals, antibacterials, anti-inflammatories, steroids, antipruritic agents,dietary supplements or emollients.

12. A pharmaceutical composition according to claim 11, wherein the antiparasitics are selected from arylpyrazoles, avermectins, milbemycins, organophosphates or pyrethroids; the antihistamines are selected from chlorpheniramine, trimeprazine, diphenhydramine or doxylamine; the antifungals are selected from fluconazole, ketoconazole, itraconazole, griseofulvin or amphotericin B; the antibacterials are selected from enroflaxacin, marbofloxacin, ampicillin or amoxycillin; the anti-inflammatories are selected from prednisolone, betamethasone, dexamethasone, carprofen or ketoprofen; and the dietary supplement is gamma-linoleic acid.

13. A method of treating periodontal disease or an opportunistic bacterial infection in or on a dog or cat, resulting from (a) a compromised or diminished immunological system, (b) Feline Immunodeficiency Virus (FIV) or (c) cancer chemotherapy in or on a comprising administrating a therapeutically effective amount of a compound of Formula I 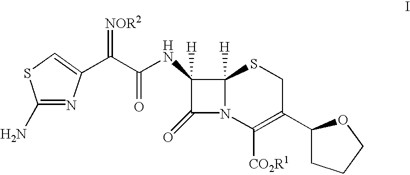
wherein R¹ is H, a pharmaceutically acceptable cation salt, or an in vivo hydrolysable group capable of generating CO₂H, and R² is C₁₋₄ alkyl, effective in treating such a condition.

14. A method according to claim 13, wherein the pharmaceutically acceptable cation salt is Na⁺, K⁺ or Li⁺.

15. A method of treating a disease or condition caused by a bacterial infection in or on a dog or cat comprising administrating a therapeutically effective amount of a compound of Formula I, 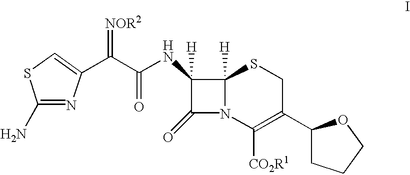
wherein R¹ is H, a pharmaceutically acceptable cation salt, or an in vivo hydrolysable group capable of generating CO₂H, and R² is C₁₋₄ alkyl, and a pharmaceutical diluent or carrier, with the proviso that if the pharmaceutically acceptable cation salt is Na, Li or K, R² is not methyl, effective in treating such a condition.

16. A method according to claim 15, wherein the disease or condition is a skin, soft tissue or urinary tract bacterial infection.

17. A method according to claims 13, 14, 15 or 16, wherein the composition is administered to the dog or cat in a single dose.

18. A method according to claim 17, wherein administration is subcutaneous.

19. A method according to claim 18, wherein the therapeutically effective amount of the compound of Formula I is 4 mg/kg to 12 mg/kg.

20. A method according to claim 19, wherein the method provides a duration of treatment activity of at least five days against susceptible pathogens.

21. A method according to claim 20, wherein the duration is at least 7 days.

22. A method of treating a disease or condition caused by a bacterial infection in or on a dog or cat comprising administrating a therapeutically effective amount of the pharmaceutical composition according to claim 9.

23. A kit for treatment or prevention of a bacterial infection or a condition caused or complicated by a bacterial infection, in or on a dog or cat, comprising:
a) a pharmaceutical composition according to claims 1, 2, 3, 4 or 5; and
b) instructions describing a method of using the pharmaceutical composition to treat a bacterial infection or a condition caused or complicated by a bacterial infection, in or on a dog or cat.

24. A method for increasing acute or chronic injection-site toleration in a dog or a cat, comprising administering to a dog or a cat in need thereof a single dose of a therapeutically effective amount of a Formula I 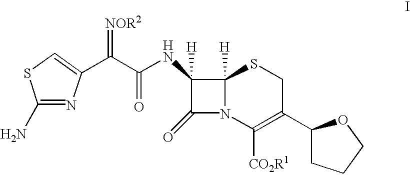
wherein R¹ is H, a pharmaceutically acceptable cation salt, or an in vivo hydrolysable group capable of generating CO₂H, and R² is C₁₋₄alkyl, effective in treating such a condition.
